(19) United States
(12) Patent Application Publication
(10) Pub. No.: US 2012/0028934 A1
(43) Pub. Date: Feb. 2, 2012

(54) STABLE PHARMACOLOGICALLY ACTIVE COMPOSITIONS INCLUDING VITAMIN D-CONTAINING AND CORTICOSTEROID COMPOUNDS WITH LOW pH COMPATIBILITY

(76) Inventors: Rakefet COHEN, Zur Yigal (IL); Michael Fox, Tel Aviv (IL)

(21) Appl. No.: 13/271,890

(22) Filed: Oct. 12, 2011

Related U.S. Application Data
(62) Division of application No. 11/897,540, filed on Aug. 29, 2007.
(60) Provisional application No. 60/841,164, filed on Aug. 29, 2006.

(51) Int. Cl. A61K 31/59; A61P 17/06; A61K 31/593

(52) U.S. Cl. 514/167

(57) ABSTRACT

Provided are pharmaceutical compositions comprising at least one vitamin D-containing compound, at least one corticosteroid compound, and at least one solvent component selected from the group consisting of triglycerides, sorbitan, sorbitan fatty esters, cetearyl glucoside, PEG-n sorbitan stearate, acrylamide/sodium acryloyldimethyl taurate copolymers, and mixtures thereof. Also provided are methods of making such compositions, and methods for treating psoriasis using such compositions.

### CROSS-REFERENCE TO RELATED APPLICATIONS

This application claims the benefit of U.S. Ser. No. 60/841,164, filed Aug. 29, 2006, which is incorporated herein by reference in its entirety.

### FIELD OF INVENTION

The present invention encompasses compositions containing, for example, a vitamin D-containing compound and a corticosteroid compound.

### BACKGROUND OF THE INVENTION

Vitamin D is a fat-soluble vitamin. It is found in food, but also can be made in the body after exposure to ultraviolet rays. Vitamin D is known to exist in several chemical forms, each with a different activity. Some forms are relatively inactive in the body, and have limited ability to function as a vitamin. The liver and kidney help convert vitamin D to its active hormone form. The major biologic function of vitamin D is to maintain normal blood levels of calcium and phosphorus. Vitamin D aids in the absorption of calcium, helping to form and maintain healthy bones. The structure of 1α,24(S)-dihydroxy vitamin D₂ is shown below:

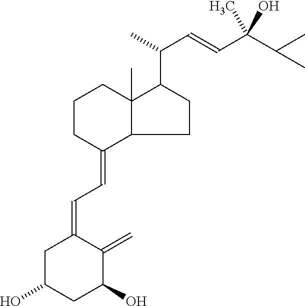

Betamethasone dipropionate dipropionate (9-Fluoro-11β,17,21-trihydroxy-16β-methylpregna-1,4-diene-3,20-dione17,21-dipropionate) is a topical corticosteroid. It has anti-inflammatory, antipruritic, vasoconstrictive and immunosuppressive properties, however, without curing the underlying condition. The mechanism of the anti-inflammatory activity of the topical steroids, in general, is unclear. Clinical studies with radiolabelled ointment indicate that the systemic absorption of betamethasone from the reference product DOVOBET is less than 1% of the dose when applied to normal skin for 12 hours. The product is white to almost white powder. The structure of Betamethasone dipropionate is shown below:

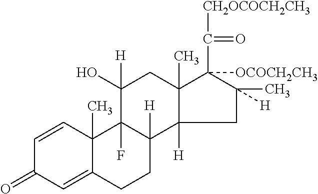

Topical steroid compounds, such as corticosteroids, and vitamin D-containing or vitamin D-containing analogues, such as calcipotriene (calcipotriol), are used to treat psoriasis or other inflammatory diseases. Topical corticosteroids and calcipotriene have been used separately for the treatment of psoriasis. Clearly, it would be useful to combine vitamin D-containing analogues such as calcipotriene and a corticosteroid in the same treatment in order to avoid the need for separate applications.

However, simultaneous application of the two products apparently is not recommended due to reported incompatibility between the currently marketed corticosteroid and calcipotriene formulations. These two classes of compounds often have specific optimum stability pH values which differ significantly from one another. For example, the vitamin D-containing analogue calcipotriene, similar to other members of its class, is reportedly most stable at a pH greater than about 8. On the other hand, betamethasone, like other corticosteroids, is reportedly most stable at a pH in the range of about 4 to about 6. As a result of the different maximum stability pH values, formulating a stable topical preparation containing a steroid compound and a vitamin D-containing analogue can present a challenge. Moreover, excipients traditionally used in the preparation of topical formulations such as creams or ointments are often acidic or alkaline in nature, causing the combination of the two active components to be potentially unstable.

The polymorphic form of the vitamin D-containing analogue has also been reported to affect stability. U.S. Pat. No. 5,763,426 asserts that calcipotriol hydrate is “surprisingly stable”.

U.S. Pat. No. 6,753,013 describes a pharmaceutical composition for dermal use including a combination of a vitamin D-containing analogue and a corticosteroid, admixed with a solvent component (generally an ether or alcohol)wo compounds to coexist despite differing pH stabiliy profiles. However, all working examples and specifically disclosed embodiments in the '013 patent disclose that the calcipotriol used is the (reportedly) more “stable” hydrate form. The anhydrous form is not mentioned in the examples and is less stable.

Further, PCT Publication 02/34235 notes that esters are generally not compatible with Vitamin D noting that some vitamin D analogues tend to be degraded in the presence of even small amounts of free fatty acids found as impurities in esters, and suggesting that preferred surfactants for inclusion in composition comprising such vitamin D analogues are therefore ethers.

Additionally, EP Publication 0679154 discloses a hydrated crystalline form of calcipotriol that is stated to have enhanced stability as compared with the anhydrous form.

There is a need in the art to provide a pharmaceutical composition containing a vitamin D-containing analogue and a corticosteroid compound that is stable without regard to the state of hydration of the vitamin D-containing analogue.

### SUMMARY OF THE INVENTION

As used herein, the term “stable” refers to an active compound which remains within +/−10%, preferably 6%, by weight, of the original amount, when incubated at the recited temperature for the recited amount of time in a closed container.

As used herein, the term stiffening agent refers to a compound which, when added to the composition, will impart a rigidity to it.

As used herein, the term “anhydrite” means any compound free of the water of hydration, as would be understood in the art.

As used herein, the term “medium chain triglycerides” refers to triglycerides of saturated fatty acids, such as of caprylic acid (octanoic acid, C₈H₁₆O₂) and capric acid (decanoic acid, C₁₀H₂₀O₂), which can be obtained from the hard, dried fraction of the endosperm of Cocos nucifera L. or the dried endosperm of Elaeis guineensis Jacq, and have a minimum of 95% of saturated fatty acids with 8 and 10 carbon atoms.

This invention presents stable compositions comprising a vitamin D-containing compound analogues and a corticosteroid compound in a solvent (or mixture of solvents), which compositions are suitable for topical applications.

Preferably, the vitamin D-containing-containing compound includes calcipotriene, and more preferably calcipotriene anhydrate. Preferably, the vitamin D-containing compound includes at least about 50% calcipotriene anhydrate by weight, and results in a preferred concentration of calcipotriene anhydrate of 0.1-0.001% (by weight) of calcipotriene anhydrate in the final product.

Preferably, the corticosteroid compound includes betamethasone, and more preferably betamethasone dipropionate, at a concentration of 0.1-0.01% (by weight) in the final product. Preferably, the composition includes both calcipotriene anhydrate and betamethasone dipropionate.

Additionally, the solvent component includes at least one of a medium chain (preferably 6-12 carbon atoms) fatty acid esters of glycerol, triglyceride or polysorbate. Preferably, the composition includes at least one of an antioxidant, a stiffening agent (an oil matrix forming agent), or a preservative such as tocopherol, BHT, or BHA.

Preferably, the composition has at least one of the following stability profiles:

(a) the amount of the vitamin D-containing compound and corticosteroid compound in the composition measured by a quantitative assay is stable (within +/−10%, preferably 6%, of the original amount) when the composition is stored at 40° C. for one month, preferably three months; and/or

(b) the amount of the vitamin D-containing compound and corticosteroid compound in the composition as measured by a quantitative assay is stable (as defined above) when the composition is stored at 55° C. for 3 days.

In both of the above cases, stability is measured after incubation in a closed container at the recited temperature for the recited amount of time; stability is determined by any quantitative assay for the recited component, and is preferably determined by HPLC methodologies known in the art.

### DETAILED DESCRIPTION OF THE INVENTION

Preferred embodiments of the invention provide compositions including a solvent component where a vitamin D-containing compound and corticosteroid compound co-exist without degradation, even when the vitamin D-containing compound, e.g., calcipotriene, is an anhydrate.

In one embodiment the invention provides pharmaceutical compositions which avoid the inconvenience of a two-step or multi-step regimen for the treatment of psoriasis or other inflammatory diseases. Such a composition increases patient compliance and substantially improves the quality of life for psoriatic patients. In addition, stable compositions that can utilize the anhydrate form of calcipotriol will provide further options to formulators in their choice of active ingredients.

In another embodiment, the present invention provides a pharmaceutical composition for topical use including at least one vitamin D-containing compound, at least one corticosteroid compound, and at least one solvent component selected from the group consisting of triglycerides, sorbitan, sorbitan fatty esters, cetearyl glucoside, PEG-n sorbitan stearate, acrylamide/sodium acryloyldimethyl taurate copolymers, and mixtures thereof.

In one embodiment, the present invention encompasses a pharmaceutical composition for topical use including at least one vitamin D-containing compound, at least one corticosteroid compound, and at least one solvent component selected from the group consisting of triglycerides, preferably Miglyol™ 810, Miglyol™ 812, Myritol™ 318 (Caprylic/Capric Acid triglyceride mixtures], sorbitan, and sorbitan fatty esters such as Sorbitan monostearate, cetearyl glucoside, PEG-n sorbitan stearate, acrylamide/sodium acryloyldimethyl taurate copolymers, and mixtures thereof. As used herein, the term “medium chain triglycerides” refers to mixtures of triglycerides of saturated fatty acids, such as of caprylic acid (octanoic acid, C₈H₁₆O₂) and capric acid (decanoic acid, C₁₀H₂₀O₂), which can be obtained from the hard, dried fraction of the endosperm of Cocos nucifera L. or the dried endosperm of Elaeis guineensis Jacq, and have a minimum of 95% of saturated fatty acids with 8 and 10 carbon atoms.

As used herein, the term “vitamin D-containing compound” includes vitamin D, its prodrugs, natural or synthetic analogues, and crystalline forms including anhydrate, hydrate, solvate, or amorphous forms. Preferred vitamin D-containing compounds include calcipotriene (calcipotriol), calcitriol, tacalcitol, maxacalcitol, or 1(S),3(R)-dihydroxy-20(R)-[((3(2-hydroxy-2-propyl)-phenyl)-methoxy)-methyl]-9,10-seco-pregna-5(Z),7(E),10(19)-triene. Preferably, the vitamin D-containing compound is calcipotriene, and more preferably calcipotriene anhydrate. Also preferably, the vitamin D-containing compound includes at least about 50% calcipotriene anhydrate by weight.

Preferably, the vitamin D-containing compound in such a composition is calcipotriene anhydrate. As used herein, the term “anhydrate” means not having water of hydration. More preferably, the vitamin D-containing compound in such a composition includes at least about 50% (more preferably at least about 70% and even more preferably at least about 90%) calcipotriene anhydrate by weight as measured by any quantitative assay known in the art. A preferred assay is the use of HPLC and comparison against standard solutions.

More preferably, the vitamin D-containing compound includes at least about 50% calcipotriene anhydrate by weight. Preferably, the corticosteroid compound includes betamethasone, and more preferably betamethasone dipropionate. Preferably, the solvent component includes at least one of a medium chain triglyceride or polysorbate. Preferably, the method further includes combining at least one of an antioxidant, a stiffening agent, or a preservative.

Preferred corticosteroid compounds include betamethasone (9-fluoro-11,17,21-trihydroxy-16-methylpregna-1,4-diene-3,20-dione) and esters thereof such as the 21-acetate, 17-adamantoate, 17-benzoate, 17-valerate, and 17,21-dipropionate; alclomethasone and esters thereof such as the dipropionate; clobetasole and esters thereof such as the propionate; clobetasone and esters thereof such as the 17-butyrate; desoximetasone; diflucortolone and esters thereof, diflorasone and esters thereof such as the diacetate; fluocinonide; flumetasone and esters thereof such as the pivalate; fluocinolone and ethers and esters thereof such as the acetonide; fluticasone and esters thereof such as the propionate; fluprednidene and esters thereof such as the acetate; halcinonide; hydrocortisone and esters thereof such as the −17-butyrate; mometasone and esters thereof such as the furoate; and triamcinolone and ethers and esters thereof such as the acetonide. Betamethasone or esters thereof such as the valerate or dipropionate are preferred.

The solvent component preferably includes at least one of triglyceride, sorbitan, sorbitan fatty ester, cetearyl glucoside, PEG-n sorbitan stearate, or acrylamide/sodium acryloyldimethyl taurate copolymer. Preferably, the solvent component at least one of medium chain (preferably 6-12 carbon atoms) fatty acid esters of glycerol, triglyceride or polysorbate.

Preferably, the compositions of the present invention further include at least one of the composition includes at least one of an antioxidant, a stiffening agent (an oil matrix forming agent), or a preservative such as tocopherol, BHT, or BHA.

In a preferred embodiment, the composition includes calcipotriene anhydrate, betamethasone dipropionate, paraffin, medium chain triglyceride, and tocopherol.

In another preferred embodiment, the composition includes calcipotriene anhydrate, betamethasone dipropionate, paraffin, polysorbate, and tocopherol. In preferred embodiments, the assay of the vitamin D-containing compound and corticosteroid compound in the composition is stable (as defined above) when the composition is stored at 40° C. for one month, preferably three months, and the assay of the vitamin D-containing compound and corticosteroid compound in the composition is stable (as defined above) when the composition is stored at 55° C. for 3 days. In the same or other preferred embodiments, the assay of the vitamin D-containing compound and corticosteroid compound in the composition is about the same when the composition is stored at 40° C. for one month, preferably three months, and the assay of the vitamin D-containing compound and corticosteroid compound in the composition is stable (as defined above) when the composition is stored at 55° C. for 3 days.

In a preferred embodiment, compositions of the present invention have at least one of the following stability profiles:

(a) the amount of the vitamin D-containing compound and corticosteroid compound in the composition measured by a quantitative assay is about the same (within +/−10%, preferably 6%, of the original amount) when the composition is stored at 40° C. for one month; preferably three months, and/or

(b) the amount of the vitamin D-containing compound and corticosteroid compound in the composition as measured by a quantitative assay is about the same (as defined above) when the composition is stored at 55° C. for 3 days.

In another embodiment, the present invention provides a method for preparing a pharmaceutical composition for topical use including combining at least one vitamin D-containing compound, at least one corticosteroid compound, and at least one solvent component selected from the group consisting of triglycerides, sorbitan, sorbitan fatty esters, cetearyl glucoside, PEG-n sorbitan stearate, acrylamide/sodium acryloyldimethyl taurate copolymer, and mixtures thereof to form the composition.

The compositions of the present invention may be prepared in accordance with methods well known to skilled person. In a preferred embodiment, the method includes dissolving the vitamin D-containing compound in at least one solvent component, and combining with the corticosteroid compound.

In a preferred embodiment, the method includes preparing a mixture of the vitamin D-containing compound, the solvent component, and paraffin; preparing a mixture of the corticosteroid compound and mineral oil (or similar substance that aids in the dispersal of the paraffin matrix homogeneously throughout the composition and/or contributes to the ability to apply the subsequent composition as an even layer to the desired target); and combining the mixtures to form the composition.

Preferably, the method includes preparing a mixture of calcipotriene, at least one of a medium chain triglyceride or polysorbate, and melted paraffin; preparing a mixture of betamethasone dipropionate, tocopherol and paraffin; and combining the mixtures to form the composition.

Preferably, the method produces compositions where the assay of the vitamin D-containing compound and corticosteroid compound in the composition is stable (as defined above) when the composition is stored at 40° C. for one month, preferably three months, and the assay of the vitamin D-containing compound and corticosteroid compound in the composition is stable (as defined above) when the composition is stored at 55° C. for 3 days. Also preferably, the assay of the vitamin D-containing compound and corticosteroid compound in the composition is about the same when the composition is stored at 40° C. for one month, preferably three months, and the assay of the vitamin D-containing compound and corticosteroid compound in the composition is stable (as defined above) when the composition is stored at 55° C. for 3 days.

In another embodiment, the present invention provides a method for preparing a pharmaceutical composition for topical use including combining at least one vitamin D-containing compound, at least one corticosteroid compound, and at least one solvent component selected from the group consisting of triglycerides, sorbitan, sorbitan fatty esters, cetearyl glucoside, PEG-n sorbitan stearate, acrylamide/sodium acryloyldimethyl taurate copolymers, and mixtures thereof to form the composition, preferably triglycerides and sorbitan, more preferably triglycerides.

In one embodiment, the method includes dissolving the vitamin D-containing compound in at least one solvent component, and combining the solution with a corticosteroid compound.

Further, the method preferably includes preparing a mixture of the vitamin D-containing compound, the solvent component, and paraffin; preparing a mixture of the corticosteroid compound and mineral oil (or similar substance that aids in the dispersal of the paraffin matrix homogeneously throughout the composition and/or contributes to the ability to apply the subsequent composition as an even layer to the desired target); and combining the mixtures to form the composition. Preferably, the calcipotriene is an anhydrate.

In one embodiment, the method includes preparing a mixture of calcipotriene, at least one of a medium chain (preferably 6-12 carbon atoms) fatty acid esters of glycerol, triglyceride or polysorbate, and melted paraffin; preparing a mixture of betamethasone dipropionate, tocopherol and paraffin; and combining the mixtures to form the composition. Preferably, the calcipotriene is an anhydrate.

Preferably, the method produces compositions exhibiting the claimed stability profiles of the invention.

In another embodiment, the present invention encompasses a method for treating psoriasis including administering to a patient in need thereof using the compositions of the present invention.

In a preferred embodiment the invention provides a topical pharmaceutical composition in the form of an ointment, a cream, a lotion, preferably a scalp lotion, a liniment or other spreadable liquid or semi liquid preparation which is, preferably, non-aqueous or in the form of an oil-in-water or water-in-oil emulsion. In one preferred embodiment, the composition of the invention is a mono-phase composition, i.e., a composition including a single solvent system, such as an ointment.

In addition to the components described above, the pharmaceutical compositions of the present invention may further contain one or more excipients. Selection of excipients and the amounts to use may be readily determined by the formulation scientist based upon experience and consideration of standard procedures and reference works in the field. Preferred examples of such excipients include stiffening agents such as microcrystalline wax and hard paraffin; antioxidants such as tocopherol, butylated hydroxyanisole, and butylated hydroxytoluene; and preservatives such a parabens, preferably butylparaben and propylparaben.

In another embodiment, the present invention encompasses a method for treating psoriasis including administering to a patient in need thereof using the compositions of the present invention.

While it is apparent that the invention disclosed herein is well calculated to fulfill the objects stated above, it will be appreciated that numerous modifications and embodiments may be devised by those skilled in the art. Therefore, it is intended that the appended claims cover all such modifications and embodiments as falling within the true spirit and scope of the present invention.

### EXAMPLES

### Example 1

### Caleipotriene and Betamethasone Dipropionate Ointment

An ointment containing calcipotriene and betamethasone dipropionate was prepared as follows:

| Ingredient | Quantity (% W/W) |
|---|---|
| White soft paraffin | 91.929 |
| Medium chain triglyceride | 5.000 |
| Calcipotriene (anhydrate) | 0.005 |
| Paraffin liquid heavy | 3.000 |
| DL-alpha-tocopherol | 0.002 |
| Betamethasone dipropionate | 0.064 |

1. 1378.93 g of white soft paraffin was melted at about 80° C., followed by cooling to about 70° C. The melted paraffin was saturated with nitrogen and maintained at this temperature.

2. 75 mg of calcipotriene (anhydrite) was dissolved in 75 g preheated medium chain triglyceride (myritol 318), saturated with nitrogen.

3. 30 mg of tocopherol was dissolved in 45 g of paraffin liquid.

4. 965 mg of betamethasone dipropionate was dispersed in the liquid from step 3.

5. The solution from step 2, containing calcipotriene was added slowly to the melted white soft paraffin while stirring, under nitrogen protection.

6. The dispersion from step 4 was added to the calcipotriene containing mixture from step 5 while stirring, under nitrogen protection.

7. The mixture was cooled down to below 30° C. while stirring, under nitrogen protection.

The amount of the recited components in Table 1 were measured by quantitiative HPLC 1-2 days after the cooling (Time zero) and at the recited times after storage at the recited temperatures.

TABLE 1
Stability of the calcipotriene and betamethasone dipropionate
composition at 40° C.

| Assay (%) | Time zero | 1 months | 2 months | 3 months |
|---|---|---|---|---|
| Calcipotriene | 95.8 | 98.0 | 97.1 | 100.5 |
| Betamethasone | 93.5 | 100.35 | 98.7 | 103.6 |
| Impurities/degradants | 0.15 | 0.27 | ND | ND |

TABLE 2
Stability of the calcipotriene and betamethasone dipropionate
composition at 55° C.

| Assay (%) | Time zero | 3 days |
|---|---|---|
| Calcipotriene | 95.8 | 96.4 |
| Betamethasone | 93.5 | 94.5 |
| Impurrties/degradants | 0.1.5 | 0.12 |

### Example 2

### Caleipotriene and Betamethasone Dipropionate Ointment

An ointment containing calcipotriene and betamethasone dipropionate was prepared as follows:

| Ingredient | Quantity (% W/W) |
|---|---|
| White soft paraffin | 91.929 |
| Polysorbate 80 | 5.000 |
| Calcipotriene (anhydrous) | 0.005 |
| Paraffin liquid heavy | 3.00 |
| DL-alpha-tocopherol | 0.002 |
| Betamethasone dipropionate | 0.064 |

1. 1378.93 g of white soft paraffin was melted at about 80° C., followed by cooling to about 70° C. The melted paraffin was saturated with nitrogen and maintained at this temperature.
2. 75 mg of calcipotriene (anhydrate) was dissolved in 75 g preheated polysorbate 80, saturated with nitrogen.
3. 30 mg of tocopherol was dissolved in 45 g of Paraffin liquid.
4. 965 mg of betamethasone dipropionate was dispersed in the liquid from step 3
5. The solution from step 2, containing calcipotriene was added slowly to the melted white soft paraffin while stirring, under nitrogen protection.
6. The dispersion from step 4 was added to the calcipotriene containing mixture from step 5 while stirring, under nitrogen protection.
7. The mixture was cooled down to below 30° C. while stirring, under nitrogen protection.
   The amount of the recited components in Table 1 were measured by quantitiative HPLC 1-2 days after the cooling (Time zero) and at the recited times after storage at the recited temperatures).

TABLE 3
Stability of the calcipotriene and betamethasone dipropionate
composition at 40° C.

| Assay | Time zero | 1 months | 2 months |
|---|---|---|---|
| Calcipotriene (%) | 99.1 | 97.9 | 97.4 |
| Betamethasone | 97.4 | 96.3 | 95.8 |
| Impurities/degradants | 0.53 | 0.91 | 1.5 |

It is apparent that many modifications and variations of this invention as hereinabove set forth may be made without departing from the spirit and scope thereof. The specific embodiments described are given by way of example only, and the invention is limited only by the terms of the appended claims.

1-14. (canceled)

15. A method for preparing a pharmaceutical composition for topical use comprising combining at least one vitamin D-containing compound, at least one corticosteroid compound, and at least one solvent component selected from the group consisting of triglycerides, sorbitan, sorbitan fatty esters, cetearyl glucoside, PEG-n sorbitan stearate, acrylamide/sodium acryloyldimethyl taurate copolymers, and mixtures thereof to form the composition.

16. The method of claim 15 wherein the vitamin D-containing compound is an anhydrate.

17. The method of claim 15, further comprising combining at least one of an antioxidant, a stiffening agent, or a preservative.

18. The method of claim 15, wherein the at least one vitamin D-containing compound comprises calcipotriene.

19. The method of claim 15, wherein the at least one vitamin D-containing compound is calcipotriene anhydrate.

20. The method of claim 15, wherein the at least one vitamin D-containing compound includes at least about 50% calcipotriene anhydrate by weight.

21. The method of claim 15, wherein the at least one corticosteroid compound comprises betamethasone.

22. The method of claim 15, wherein the at least one corticosteroid compound includes betamethasone dipropionate.

23. The method of claim 15, wherein the at least one solvent component includes at least one of a medium chain triglyceride or a polysorbate.

24. The method of claim 15, wherein the at least one vitamin D-containing compound includes calcipotriene anhydrate, and the at least one corticosteroid compound includes betamethasone dipropionate.

25. The method of claim 15 comprising dissolving the vitamin D-containing compound in at least one solvent component, and combining with the corticosteroid compound.

26. The method claim 15 further comprising the steps of:
(a) preparing a mixture of the at least one vitamin D-containing compound, the at least one solvent component, and paraffin;
(b) preparing a mixture of the at least one corticosteroid compound and mineral oil; and
(c) combining the mixtures of steps (a) and (b) to form the composition.

27. The method of claim 15 further comprising the steps of:
(a) preparing a mixture of calcipotriene, at least one of a medium chain triglyceride or polysorbate, and melted paraffin;
(b) preparing a mixture of betamethasone dipropionate, tocopherol and paraffin; and
(c) combining the mixtures of steps (a) and (b) to form the composition.

28. The method of claim 15, wherein the composition has at least one of the following stability profiles:
(a) the amount of the vitamin D-containing compound and the amount of corticosteroid compound in the composition do not decrease after the composition is stored at 40° C. for three months; or
(b) the amount of the vitamin D-containing compound and the amount of the corticosteroid compound in the composition do not decrease after the composition is stored at 55° C. for 3 days.

29. The method of claim 15, wherein:
(a) the amount of the vitamin D-containing compound and the amount of corticosteroid compound in the composition do not decrease after the composition is stored at 40° C. for three months; and
(b) the amount of the vitamin D-containing compound and the amount of the corticosteroid compound in the composition do not decrease after the composition is stored at 55° C. for 3 days.

30. A composition prepared according to the method of claim 15.

31. (canceled)
